(19) United States
(12) Patent Application Publication
(10) Pub. No.: US 2007/0066865 A1
(43) Pub. Date: Mar. 22, 2007

(54) CAPSULE RETRIEVER

(75) Inventors: Masaaki Fukuda, Tokyo (JP); Masaaki Nakashima, Tokyo (JP); Taro Nakasendo, Tokyo (JP)

Correspondence Address: GREENBLUM & BERNSTEIN, P.L.C., 1950 ROLAND CLARKE PLACE, RESTON, VA, 20191, US

(73) Assignee: PENTAX Corporation, Tokyo (JP)

(21) Appl. No.: 11/532,992

(22) Filed: Sep. 19, 2006

(30) Foreign Application Priority Data
Sep. 22, 2005 (JP) P2005-275846

(51) Int. Cl. A61B 1/00; A61B 5/05; A61B 19/02

(52) U.S. Cl. 600/102; 600/407; 206/438

(57) ABSTRACT

A capsule retriever includes a retriever and a holder. The retriever retrieves a capsule endoscope while separating the capsule endoscope from excretions, such that fluid included in the excretions can pass through the retriever. The holder holds the capsule endoscope with the retriever. The retriever is attached to the holder, so that the capsule endoscope is surrounded by the retriever and is held in the holder with the retriever, by an operation using only the holder.

### BACKGROUND OF THE INVENTION

1. Field of the Invention

The present invention relates to a capsule retriever, especially to a capsule retriever for retrieving a used capsule endoscope.

2. Description of the Related Art

Recently, a capsule endoscope device for observing inside a digestive organ has been developed. In a capsule endoscope device, a capsule endoscope for photographing inside a body is swallowed by a subject person, and images of the inside of the body of the subject person are generated by processing image data obtained by the capsule endoscope.

In a capsule endoscope device, a capsule endoscope, which is excreted from a body after photographing the inside thereof, is retrieved from excretions. The recovered capsule endoscope and a capsule retriever are preferably sealed when they are brought. Thus, a capsule recovery device, including a bag for sealing a retrieved capsule endoscope with a capsule retriever, has been used.

For recovering and sealing a capsule endoscope, a capsule retriever and a bag for sealing a capsule with the capsule retriever are necessary, and the capsule recovery device tends to become large.

Further, when using a current capsule recovery device for sealing a retrieved capsule endoscope and a retriever, operations for sealing are required, such as bending a rod type tool, moving the bent tool into a bag, and other operations. Such operations are preferably omitted because they disturb prompt recovery of a capsule endoscope, and excretions may adhere to a retrieved capsule and a retriever.

### SUMMARY OF THE INVENTION

Therefore, an objective of the present invention is to provide a capsule retriever that has a simple structure, and that enables recovery of a capsule endoscope and sealing of the retrieved capsule endoscope by a simple operation.

A capsule retriever includes a retriever and a holder. The retriever retrieves a capsule endoscope while separating the capsule endoscope from excretions, such that fluid included in the excretions can pass through the retriever. The holder holds the capsule endoscope with the retriever. The retriever is attached to the holder, so that the capsule endoscope is surrounded by the retriever and held in the holder with the retriever, by an operation of only the holder.

The holder may include a rotating member that can rotate around an axis, and the capsule endoscope may be held in the holder with the retriever by a rotation of the rotating member.

The capsule endoscope may be surrounded by the retriever and held in the holder with the retriever, by a single operation of the holder.

The capsule retriever may further include a plurality of rings provided on the edge of the retriever, and a linear member that runs through the rings, so that the retriever can have a bag shape. Further, the capsule retriever may include a ring engaging member that is provided at a mouth of the holder and that has a gate for engaging the rings. The linear member may also runs through the gate, and the linear member may be held in the holder via the mouth by an operation of the holder, so that the rings may be engaged by the ring engaging member and so that he capsule endoscope is surrounded by the bag shape retriever.

The ring engaging member may be attached to the mouth, and when a force applied to the ring engaging member by the engaged rings is larger than a predetermined amount, the ring engaging member may be detached from the mouth and be held in the holder with the capsule endoscope, the rings, and the retriever.

The holder may further comprise a cover to seal the capsule endoscope and the retriever in the holder. The retriever may include a net.

### BRIEF DESCRIPTION OF THE DRAWINGS

The present invention will be better understood from the description of the preferred embodiment of the invention set forth below, together with the accompanying drawings, in which:

FIG. 1 is a perspective view representing a capsule retriever where a capsule endoscope is captured by a net;

FIG. 2 is a perspective view representing the capsule retriever where the net and the capsule endoscope are to be held;

FIG. 3 is a perspective view representing the capsule retriever where the net and the capsule endoscope are held;

FIG. 4 is a sectional view representing the area around the mouth of the holder where the mouth is closed before recovering the capsule endoscope;

FIG. 5 is a sectional view representing the area around the mouth of the holder where the mouth is opened before recovering the capsule endoscope;

FIG. 6 is a sectional view representing the area around the mouth of the holder where the mouth is closed after recovering the capsule endoscope;

FIG. 7 is a perspective view representing the capsule retriever put on a toilet where the capsule endoscope is excreted;

FIG. 8 is a side view representing the capsule retriever shown in FIG. 7;

FIG. 9 is a view representing the capsule endoscope and the net, which are enlarged;

FIG. 10 is a perspective view where the capsule endoscope is excreted, representing the capsule retriever put on a different toilet from the toilet shown in FIG. 7; and

FIG. 11 is a side view representing the capsule retriever of FIG. 10.

### DESCRIPTION OF THE PREFERRED EMBODIMENT

Hereinafter, the embodiment of the present invention is described with reference to the attached drawings.

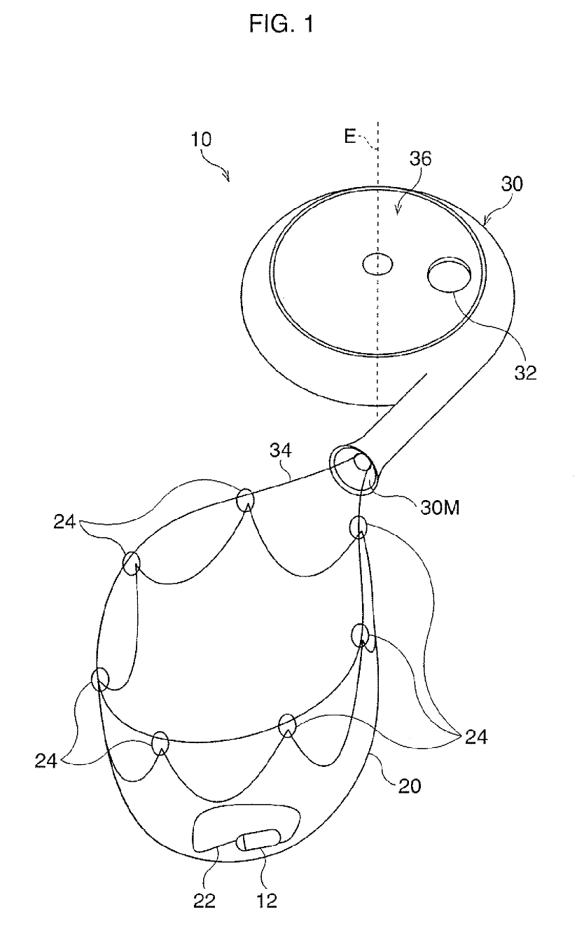

As shown in FIGS. 1 to 3, in a capsule retriever 10, a net (a retriever) 20 for recovering a capsule endoscope 12 excreted from inside a body of a subject person, and a holder 30 for holding the capsule endoscope 12 with the net 20 inside the holder 30 inside of itself are provided. The net 20 is first put on a toilet before the capsule endoscope 12 is excreted, as explained below. Then, the excreted capsule endoscope 12 and the excretions 22 are captured by the net 20 as shown in FIG. 1. The subject person washes off the shown excretions 22 with water so that the capsule endoscope 12 is separated from the excretions 22, and is left inside the net 20, which has a bag shape.

The capsule retriever 10 includes a plurality of rings 24 provided on the edge of the net 20, and an endless wire (a linear member) 34, which runs inside all the rings 24. In the holder 30, a rotating member 36 is provided. The rotating member 36 rotates about an axis E running through the center of the rotating member 36 by an operation of a user pressing and moving a recess 32 with his or her finger. The wire 34 is attached to the side surface (not shown) of the rotating member 36 inside the holder 30, via the mouth 30M of the holder 30. As it is clear from the explanation, the net 20 is attached to the holder 30 indirectly via the rings 24 and the wire 34.

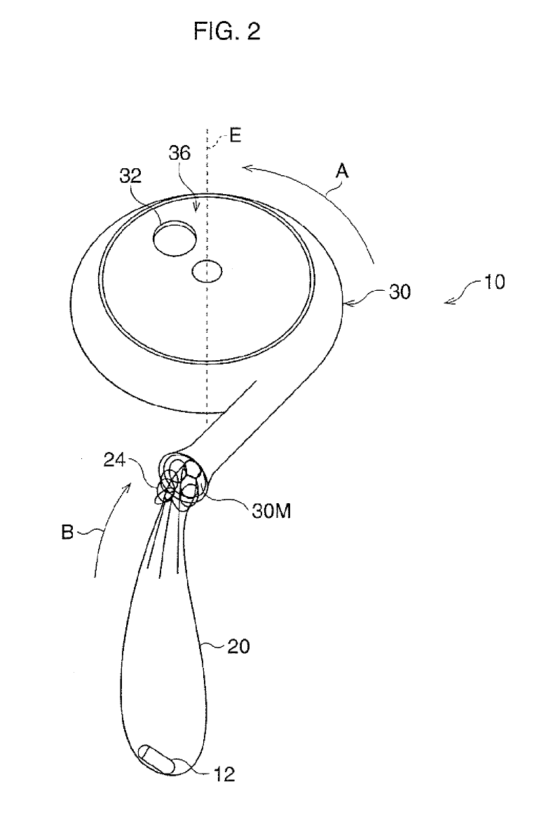

When the rotating member 36 is rotated by an operation of a subject person in a direction represented by the arrow A (see FIG. 2), the wire 34 is wound around the rotating member 36 and is gradually held inside the holder 30. At that time, the rings 24 are engaged by an engaging member (not shown here), provided around the mouth 30M and inside the holder 30. Therefore, the distances between a plurality of rings 24 are gradually shortened, the net 20 forms a bag shape with the mouth surrounded by the rings 24, the mouth is gradually closed, and the capsule endoscope 12 is surrounded by the net 20. The capsule endoscope 12 and the net 20 surrounding it are moved towards inside the holder 30 in a direction represented by the arrow 3 (see FIG. 2).

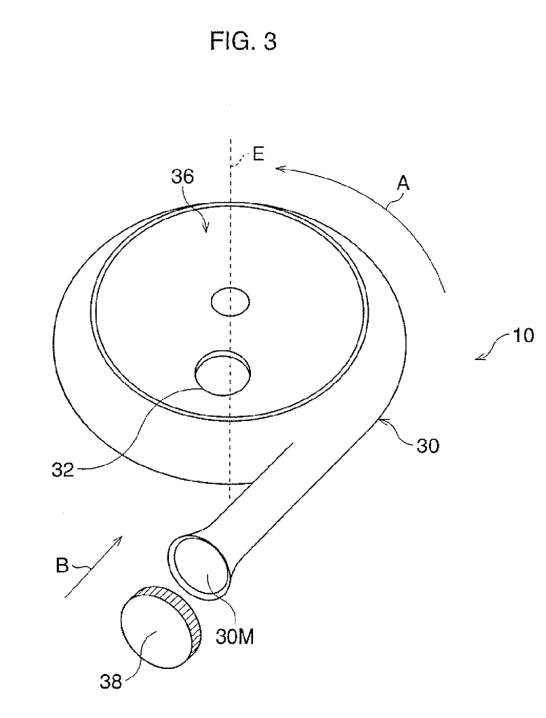

When the rotating member 36 is further rotated, the engaging member is detached from the holder 30 as explained below, and the net 20 is fully wound with the wire 34 around the rotating member 36 inside the holder 30. As a result of this, the net 20 is pulled inside the holder 30 and is held (see FIG. 3). The holder 30 includes a cover 38 for sealing the mouth 30M. By using the cover 38, the capsule endoscope 12 is sealed inside the holder 30, with the held net 20.

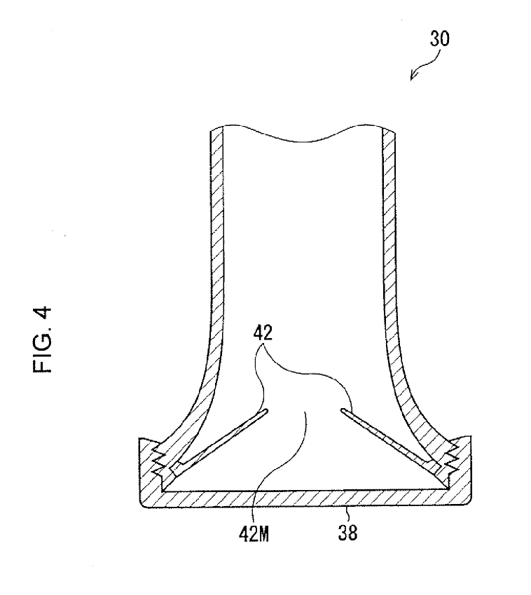

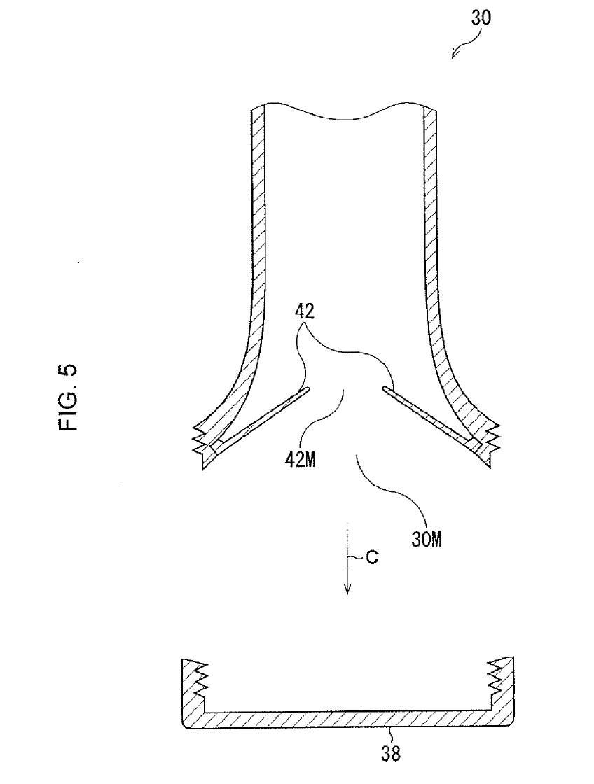

The engaging members 42 (see FIG. 4) are a plurality of projections projecting toward inside the holder 30, and are provided around the mouth 30M of the holder 30. Between the ends of these engaging members 42, a gate hole 42M, whose diameter is shorter than the outside diameter of the rings 24, is formed for engaging the rings 24. When the cover 38 is opened as the arrow C (see FIG. 5) represents, and the rotating member 36 is rotated to hold the retrieved capsule endoscope 12, the wire 34 running in the gate hole 42M is held inside the holder 30, and the rings 24 (not shown here) are engaged by the engaging members 42 (see FIG. 2). At this time, only the wire 34 is drawn inside the holder 30 via the mouth 30M first, and the rings 24 are engaged by the engaging members 42; therefore, the mouth of the net 20, having a bag shape and surrounded by the engaged rings 24, is closed so that the capsule endoscope 12 is reliably surrounded by the net 20.

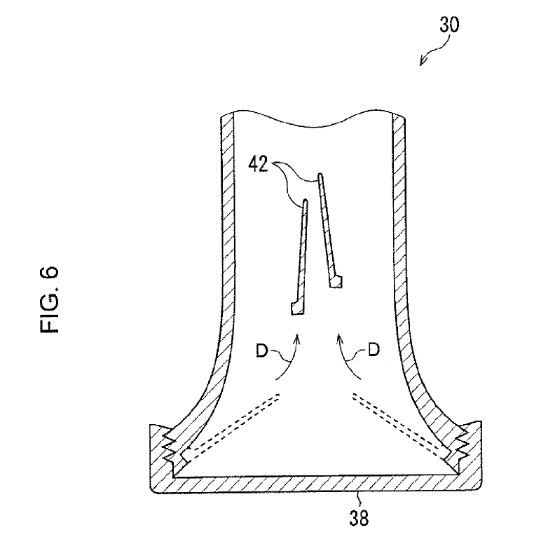

When the rotating member 36 is further rotated and all of the rings 24 are engaged by the engaging members 42, the force applied to the engaging members 42 by the engaged rings 24 becomes larger than a predetermined amount. Thus, the engaging members 42 are detached from the mouth 30M of the holder 30 (see FIG. 6) and are held inside the holder 30 with the capsule endoscope 12, the net 20 having captured the capsule endoscope 12 and the rings 24.

As explained above, the capsule endoscope 12 is surrounded by the net 20, and the capsule endoscope 12 and the net 20 are drawn inside the holder 30 (see FIG. 3) from an outside position that is separate from the holder 30 (see FIG. 1) via a single operation that rotates the rotating member 36. Therefore, a user, such as a subject person, can cause the capsule endoscope 12 and the net 20 to be held inside the holder 30 without touching the net 20 and other members by a simple operation of the holder 30.

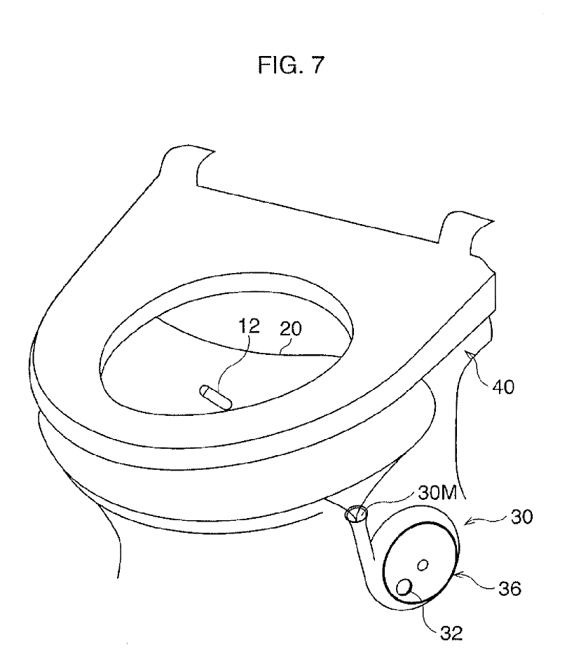

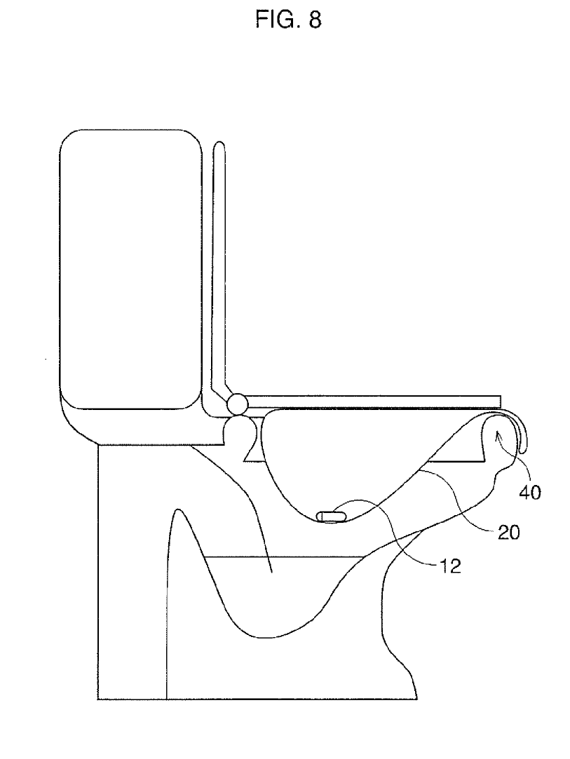

Before usage, the net 20 is pulled from inside the holder 30, and is put on a first toilet 40 (see FIGS. 7 and 8) to receive the capsule endoscope 12 excreted from the subject person. Note that in FIGS. 7 and 8, the rings 24 are omitted. After the capsule endoscope 12 is excreted, the net 20 is removed from the first toilet 40 to capture the capsule endoscope 12 (see FIG. 1).

Note that the net 20 is preferably made of a waterproof material, such as a plastic, not to be transformed or broken by fluid included in the excretions 22 or by water for washing off the excretions 22 (not shown here).

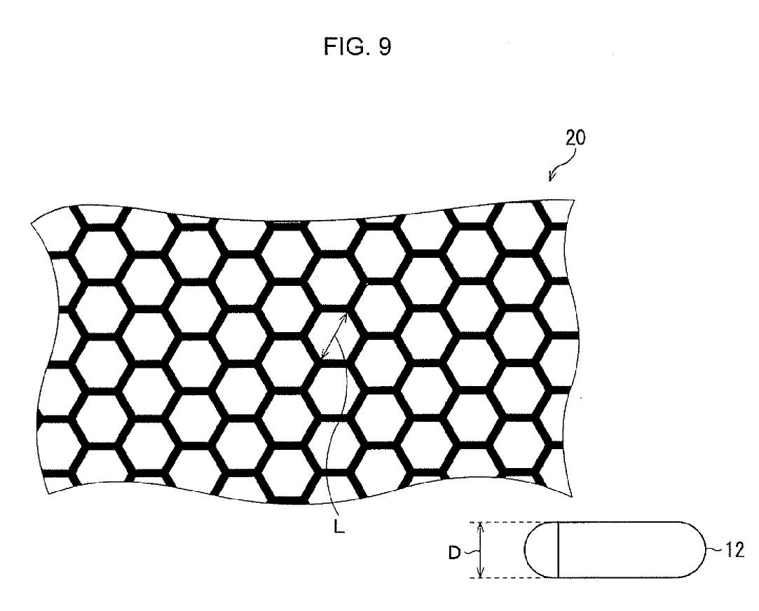

The net 20 is made of a plastic in this embodiment, and the net 20 has a honeycomb shape (see FIG. 9) with a mesh composed of many regular hexagons The length L of the diagonal of a hexagon mesh included in the net 20 is shorter than the diameter D of the capsule endoscope 12. Therefore, when the capsule endoscope 12 and the excretions 22 are captured by the net 20, only the excretions 22 is excluded, thus it is possible to leave the capsule endoscope 12 in the net 20 which forms a bag shape. That is, the capsule endoscope 12 is separated from the excretions 22 by the net 20.

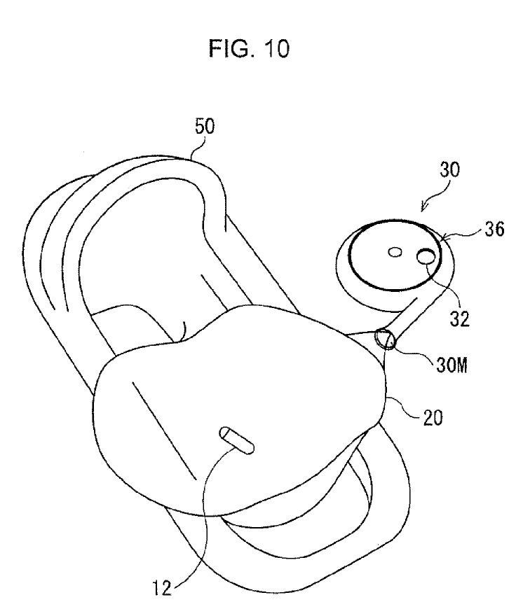

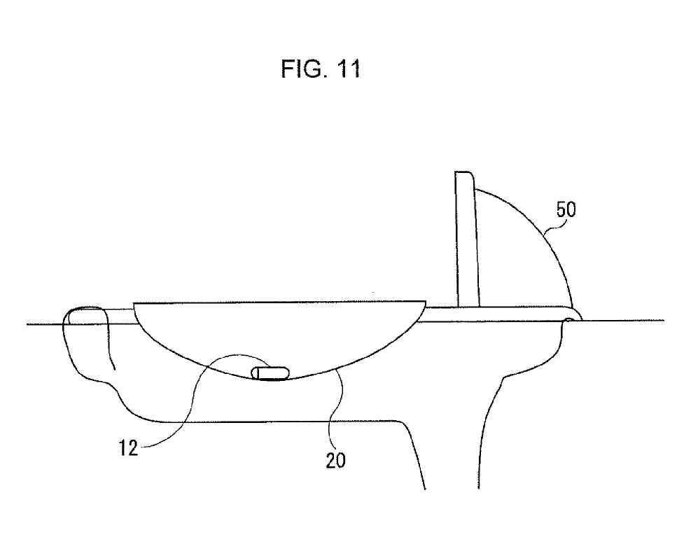

The net 20 can be used on a second toilet 50 which is different from the first toilet 40 represented in FIGS. 7 and 8 (see FIGS. 10 and 11). The size, shape, and arrangement of the net 20 can be adjusted according to the type of toilet to be used with, and the setting position on the toilet.

In this embodiment, as explained above, in the capsule retriever 10, the net 20 for recovering the capsule endoscope 12, and the holder 30 for holding the capsule endoscope 12 and the net 20 are united to each other, and the capsule retriever 10 has a simple structure.

Further, excluding the excretions 22 while capturing only the capsule endoscope 12 with the net 20 are easy, and the capsule endoscope 12 is held by an operation of the rotating member 36 in the holder 30 without touching the capsule endoscope 12 or the net 20 or other members, except for the holder 30. Therefore, the capsule retriever 10 has superior convenience, and the hands or other parts of a user do not become soiled in recovery or holding operations.

In addition to these advantages, carrying the capsule retriever 10 is easy even after usage, because the capsule endoscope 12 and the net 20 to which excretions may be attached are not exposed and completely sealed inside the holder 30.

Note that the shapes and materials of the net 20, the holder 30, and other elements are not limited to those of this embodiment. For example, the shape of each of the meshes of the net 20 is not limited to a regular hexagon. Further, instead of providing the engaging members 42, it is possible to cause the diameter of the mouth 30M to be wider than the diameter of the rings 24, and the diameter of a mouth part of the holder 30, which has a tubular shape and which is set slightly apart from the mouth 30M (see FIGS. 1 to 3), to be narrower than the diameter of the rings 24 to engage the rings 24 inside the mouth part around the mouth 30M.

On the other hand, instead of the rotating member 36, a member in which the wire 34 and the net 20 are held can be provided in the holder 30. The wire 34 may not be endless, so long as it can be wound around the rotating member 36, and the mouth of the net 20 surrounded by the rings 24 can be closed, so that the capsule endoscope 12 can be encircled by the net 20. For example, a linear wire whose ends are attached to the rotating member 36 may be used instead of the wire 34.

This invention is not limited to that described in the preferred embodiment; namely, various improvements and changes may be made to the present invention without departing from the spirit and scope thereof.

The present disclosure relates to subject matter contained in Japanese Patent Application No. 2005-275846 (filed on Sep. 22, 2005), which is expressly incorporated herein, by reference, in its entirety.

1. A capsule retriever comprising:
a retriever that retrieves a capsule endoscope while separating said capsule endoscope from excretions, such that fluid included in said excretions can pass through said retriever; and
a holder that holds said capsule endoscope with said retriever;
wherein said retriever is attached to said holder, so that said capsule endoscope is surrounded by said retriever and held in said holder with said retriever by an operation of only said holder.

2. The capsule retriever according to claim 1, wherein said holder comprises a rotating member that can rotate about an axis, and said capsule endoscope is held in said holder with said retriever by a rotation of said rotating member.

3. The capsule recovery tool according to claim 1, wherein said capsule endoscope is surrounded by said retriever and held in said holder with said retriever by a single operation of said holder.

4. The capsule retriever according to claim 1, further comprising a plurality of rings provided on the edge of said retriever, and a linear member running through said rings, so that said retriever can have a bag shape.

5. The capsule retriever according to claim 4, further comprising a ring engaging member that is provided at a mouth of said holder that has a gate for engaging said rings, wherein said linear member also runs through said gate, and said linear member is held in said holder via said mouth by an operation of said holder, so that said rings are engaged by said ring engaging member, and said capsule endoscope is surrounded by said retriever, which has a bag shape.

6. The capsule retriever according to claim 5, wherein said ring engaging member is attached to said mouth, and when a force applied to said ring engaging member by engaged said rings is greater than a predetermined amount, said ring engaging member is detached from said mouth by said force, and is held in said holder with said capsule endoscope, said rings, and said retriever.

7. The capsule retriever according to claim 1, wherein said holder further comprises a cover to seal said capsule endoscope and said retriever in said holder.

8. The capsule retriever according to claim 1, wherein said retriever comprises a net.
